(19) United States
(12) Patent Application Publication
(10) Pub. No.: US 2015/0112418 A1
(43) Pub. Date: Apr. 23, 2015

(54) Segmented Balloon Expandable Stent Graft With Reduced Foreshortening

(71) Applicant: Medtronic Vascular, Inc., Santa Rosa, CA (US)

(72) Inventor: Jeffery Argentine, Petaluma, CA (US)

(73) Assignee: Medtronic Vascular, Inc., Santa Rosa, CA (US)

(21) Appl. No.: 14/060,084

(22) Filed: Oct. 22, 2013

(51) Int. Cl. A61F 2/86; A61F 2/958

(52) U.S. Cl. CPC A61F 2/86; A61F 2/958
USPC 623/1.11; 623/1.13

(57) ABSTRACT

A segmented balloon expandable stent graft includes a graft material and a plurality of cylindrical stent elements coupled to the graft material. The plurality of cylindrical stent elements are plastically deformable when expanded from a radially compressed configuration to a radially expanded configuration. The plurality of cylindrical stent elements includes a first end stent element, a second end stent element, and a plurality of middle stent elements. The first and second end stent elements are independent of the plurality of middle stent elements. The first and second end stent elements are more resistant to radial expansion than the plurality of middle stent elements such that the plurality of middle stent elements plastically deform from the radially compressed configuration to the radially expanded configuration when inflated by a balloon prior the first and second end stent elements.

### FIELD OF THE INVENTION

The present invention relates generally to a segmented balloon expandable stent with reduced foreshortening and a method for deployed such a segmented balloon expandable stent graft.

### BACKGROUND OF THE INVENTION

Tubular prostheses, such as stents, grafts, and stent-grafts are known for treating abnormalities in various passageways of the human body. In vascular applications, these devices often are used to replace or bypass occluded, diseased or damaged blood vessels such as stenotic or aneurysmal vessels. For example, it is well known to use stent-grafts of a biocompatible graft material supported by a framework, for e.g., one or more stent or stent-like structures, to treat or isolate aneurysms. The framework provides mechanical support and the graft material or liner provides a blood barrier. When implanting a stent-graft, the stent-graft typically is placed so that one end of the stent-graft is situated proximal to or upstream of the diseased portion of the vessel and the other end of the stent-graft is situated distal to or downstream of the diseased portion of the vessel. In this manner, the stent-graft extends through and spans the aneurysmal sac and extends beyond the proximal and distal ends thereof to replace or bypass the dilated wall.

Such tubular prostheses are known to be implanted in either an open surgical procedure or by a minimally invasive endovascular/endoluminal approach. Minimally invasive endovascular stent-grafts for use in treating aneurysms are often preferred over traditional open surgery techniques where the diseased vessel is surgically opened, and a graft is sutured into position bypassing the aneurysm. The endovascular approach generally involves opening a vein or artery with a needle, inserting a guidewire into the vein or artery through the lumen of the needle, withdrawing the needle, inserting over the guidewire a dilator located inside an associated sheath introducer having a hemostasis valve, removing the dilator and inserting a delivery catheter through the hemostasis valve and sheath introducer into the blood vessel. The delivery catheter with the stent-graft secured therein may then be routed through the vasculature to the target site. For example, a stent-graft delivery catheter loaded with a stent-graft can be percutaneously introduced into the vasculature, for e.g., into a femoral artery, and the stent-graft delivered endovascularly across an aneurysm where it is then deployed.

Specialized endovascular stent-grafts have been developed for the treatment of thoracic aortic aneurysms. A thoracic aortic aneurysm a bulge that forms in the wall of the aorta in the area of the aortic arch or just below the aortic arch. Emanating from the aortic arch are three branch arteries, the innominate or brachiocephalic artery, the left common carotid artery, and the left subclavian artery. In some cases, an aneurysm in the aortic arch may extend into one of the branch arteries, or the aneurysm is located in the arch such that a main stent graft used to bypass the aneurysm will block access to the one or more of the branch arteries. Accordingly, a branch stent graft may extend through a fenestration in the main stent graft and extend into the branch artery.

However, the aortic arch represents a challenging design environment due to a significant amount of cardiac and respiratory movement. Such movement requires a branch stent graft with significant flexibility and durability to withstand such movement over an extended period of time. Further, in some cases, the fenestration of the main stent graft is not aligned with the branch artery. In such cases, the branch stent graft extends from the fenestration in the main stent graft, extends within the aorta for a short distance, and then extends into the branch artery (offset configuration). In such situations, significant flexibility is required and sufficient radial force to maintain the branch stent graft open against the force of the main stent graft while in the aorta.

Currently there are no commercially available branch stent grafts specifically designed for the aortic arch. Branch stent grafts used for other areas are not suitable for use in the aortic arch branch arteries. Known self expanding stent grafts lack the radial force required to perfuse the side branch, especially if the fenestrated aortic stent graft is deployed in an offset configuration. Known balloon expandable stent grafts are generally too stiff to decouple the large amount of motion occurring in the arch from the perfused branch vessel and these rigid stents may fracture. Accordingly, there is a need for a branch stent graft with sufficient flexibility and durability to withstand forces in the aortic arch.

Segmented balloon expandable stent grafts, such as those described in U.S. patent application Ser. No. 13/782,627, filed Mar. 1, 2013 (attorney docket no. P0039933.USU1), incorporated by reference herein in its entirety, can provide excellent flexibility and durability for a branch stent graft. However, as described therein, such segmented balloon expandable stent grafts may foreshorten when expanded by the balloon. Foreshortening results in a stent graft that, when expanded to its radially expanded configuration, is shorter than expected or desired. In such a situation, the stent graft does not cover the desired length of a treatment site, resulting in an untreated area or requiring delivery of an additional stent graft to cover the untreated area. Foreshortening may occur due to the design of the stent and the fact that the balloon is generally slightly longer than the stent graft disposed thereon. Because the stent graft resists expansion of the balloon where the stent graft is mounted on the balloon, the proximal and distal ends of the balloon tend to expand first. This pushes the proximal and distal ends of the stent graft towards each other, thereby causing foreshortening. Accordingly, there is a need for a segmented balloon expandable stent graft with sufficient flexibility and durability to withstand forces in the aortic arch and which does not foreshorten (or exhibits reduced foreshortening) during when radially expanded by a balloon.

### BRIEF SUMMARY OF THE INVENTION

Embodiments hereof are directed to a segmented balloon expandable stent graft including a graft material having a generally tubular configuration and a plurality of cylindrical stent elements coupled to the graft material. The plurality of cylindrical stent elements are plastically deformable when expanded from a radially compressed configuration to a radially expanded configuration. The plurality of cylindrical stent elements include a first end stent element disposed adjacent a first end of the graft material, a second end stent element disposed adjacent a second end of the graft material, and a plurality of middle stent elements disposed between the first end stent element and the second end stent element. The first end stent element is independent of the plurality of middle stent elements and the second end stent element is independent of the plurality of middle stent elements. Further, the first end stent element and the second end stent element are more resistant to radial expansion than the plurality of middle stent elements such that the plurality of middle stent elements plastically deform from the radially compressed configuration to the radially expanded configuration when inflated by a balloon prior the first end stent element and the second end stent element. In an embodiment, the first end stent element and the second end stent element are at least twice as resistant to radial expansion as the middle stent elements. In an embodiment the first end stent element and the second end stent element are more resistant to radial pressure by being thicker than the plurality of middle stent elements.

Embodiments hereof are also directed to a method of deploying a stent graft in a vessel. The method includes delivering the stent graft to a site within the vessel with the stent graft in a radially compressed configuration. The stent graft includes a first end portion, a second end portion, and a middle portion disposed between the first end portion and the second end portion. The stent graft includes a graft material and a plurality of independent stent elements coupled to the graft material. The method further includes radially expanding the stent graft by applying a substantially uniform radial pressure to the stent graft, wherein the independent stent elements disposed at the first end portion of the stent graft and the independent stent elements disposed at the second end portion of the stent graft expand at a higher radial pressure than the independent stent elements disposed at the middle portion of the stent graft such that the middle portion of the stent graft expands before first end portion and the second end portion. In an embodiment, the independent stent elements disposed at the first and second end portions are more resistant to radial expansion by being thicker than the independent stent elements disposed at the middle portion of the stent graft.

### BRIEF DESCRIPTION OF DRAWINGS

The foregoing and other features and advantages of the invention will be apparent from the following description of embodiments thereof as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a side view of a stent graft in accordance with an embodiment hereof.

FIG. 2 is a cross-section taken along lines 2-2 of FIG. 1.

FIG. 3 is a schematic view of a stent graft in accordance with an embodiment hereof.

FIG. 4 is a schematic view of a stent graft in accordance with an embodiment hereof.

FIG. 5 is a schematic illustration of a balloon catheter with a stent graft mounted thereon in accordance with an embodiment hereof.

FIG. 6 is a schematic view of a distal portion of the balloon catheter of FIG. 5.

FIGS. 7-11 are schematic illustrations depicting a method of deploying a segmented balloon expandable stent graft of the present application using a balloon catheter.

FIG. 12 is a schematic illustration of stent grafts of embodiments hereof disposed in the renal arteries.

FIG. 13 is a schematic illustration of stent grafts of embodiments hereof disposed in the aortic arch and branch arteries of the aortic arch.

### DETAILED DESCRIPTION OF THE INVENTION

Specific embodiments of the present invention are now described with reference to the figures, wherein like reference numbers indicate identical or functionally similar elements. Regarding “proximal” and “distal” positions referenced herein, a proximal end of a prosthesis, e.g., stent-graft, is the end closest to the heart by way of blood flow path whereas a distal end of the prosthesis is the end furthest away from the heart during deployment. In contrast, a distal end of the stent-graft delivery system or other associated delivery apparatus is usually identified as the end that is farthest from the operator, while a proximal end of the delivery system and devices is the end nearest the operator or handle of the catheter.

The following detailed description is merely exemplary in nature and is not intended to limit the invention or the application and uses of the invention. Although the descriptions of embodiments hereof are in the context of treatment of blood vessels such as the aorta and branch vessels that emanate therefrom, the invention may also be used in any other body passageways where it is deemed useful. Furthermore, there is no intention to be bound by any expressed or implied theory presented in the preceding technical field, background, brief summary or the following detailed description.

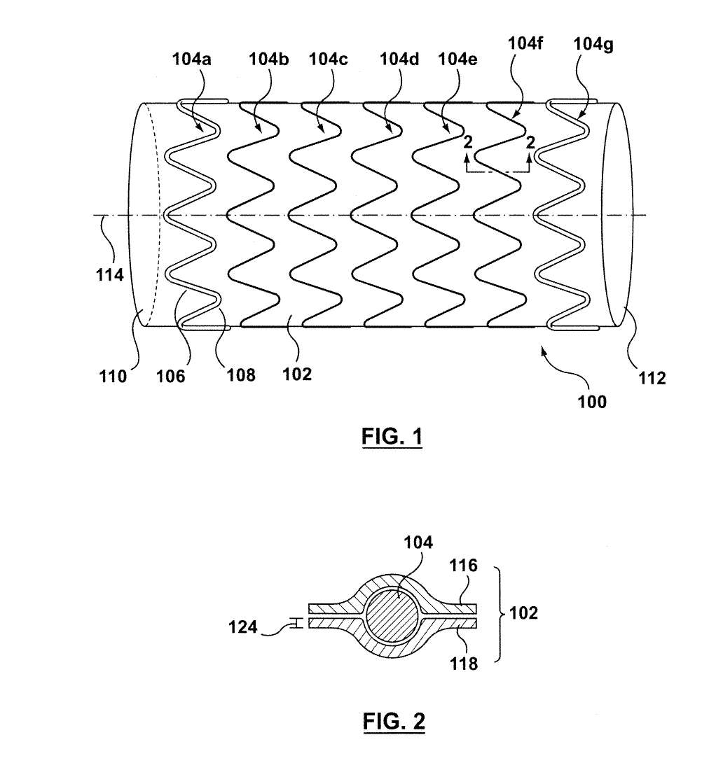

FIG. 1 is a side view of a stent graft 100 in accordance with an embodiment hereof. Stent graft 100 includes a graft material 102 and a plurality of stents 104 coupled to graft material 102. Stent graft 100 includes a first end 110 and a second end 112. Stent graft 100 is formed in a tubular shape to form a lumen therethrough and including a longitudinal axis 114, as known in the art.

In order for stent graft 100 to have the desired characteristics of flexibility and durability, graft material 102 is expanded Polytetrafluoroethylene (hereinafter “ePTFE”). Stents 104a-104g are individual rings with a zig-zag or generally sinusoidal shape including a plurality of generally straight segments or struts 106 with adjacent struts connected to each with bends or crowns 108. Although seven (7) stents 104a-104g are shown in FIG. 1, those skilled in the art would recognize that more or less stents 104 may be used. The stents 104a-104g of stent graft 100 are “segmented” in that the stents are not connected to each other except through the graft material 102. In other words, other than the graft material, other structures, such as longitudinal connectors, do not connect the stents 104a-104g to each other. Such a segmented stent graft 100 improves flexibility of the stent. However, in some instances, it may be acceptable to couple two or three adjacent stents together provided that these coupled stents are segmented from the other stents. For example, and not by way of limitation, in FIG. 1, the left-most two stents 104a and 104b could be coupled, then there would be no coupling between the second stent 104b and the third stent 104c. The third stent 104c and fourth stent 104d could be coupled together with no coupling between the fourth stent 104d and the fifth stent 104e. Further, the coupling could be with a weak or frangible connector such that after deployment, the connector breaks to de-couple the stents from each other. Accordingly, stents 104a-104g are not coupled to each other in the radially expanded configuration. Stents 104a-104g are made from a plastically deformable material such that when expanded by a balloon, the stents 104a-104g maintain their radially expanded configuration. Stents 104a-104g may be made from stainless steel, nickel-titanium alloys, cobalt-chromium alloys, tantalum alloys, various types of polymers or other materials known to those skilled in the art, including said materials coated with various surface deposits to improve clinical functionality. In an embodiment stents 104a-104g are made from stainless steel.

Stents 104a-104g are coupled to graft material 102 by being sandwiched between layers of graft material 102, as shown in FIG. 2. In particular, graft material comprises a first layer 116 and a second layer 118. Although shown as individual layers in FIG. 2, first layer 116 and second layer 118 of graft material 102 are fused together. Further, although two layers 116, 118 are shown, those skilled in the art would understand that each layer 116, 118 may be formed of several layers of expanded polytetrafluoroethylene (ePTFE). Further, ends of layers 106, 108 of graft material 102 may be folded over as described in U.S. patent application Ser. No. 13/782,627, filed Mar. 1, 2013 (attorney docket no. P0039933.USU1), incorporated by reference herein in its entirety. Although ePTFE is the preferred graft material, other graft materials may be used. For example, and not by way of limitation, graft material 102 may be a low-porosity woven or knit polyester (Dacron®) material, polytetrafluoroethylene (PTFE), polyurethane, silicone, ultra high molecular weight polyethylene, or other suitable materials. Further, stents 104a-104g may be coupled to graft material 102 in other ways known to those skilled in the art, such as stitching.

In a particular embodiment of a stent graft approximately 3.5 millimeters in diameter, layers 116, 118 of graft material 102 each have a thickness 124 of approximately 0.004 inch and have a density of approximately 0.65 grams/cubic centimeter. Those skilled in the art will recognize that the specifications for materials discussed above are exemplary and other dimensions, thicknesses, sizes, spacing, etc. may be used.

As described above, stent grafts, and in particular “segmented” stent grafts wherein the stents are not connected to each other, may experience foreshortening when expanded. In the embodiment of FIGS. 1-2, stents 104a and 104g, i.e., the stents adjacent ends 110 and 112 of stent graft 100, are more resistant to radial expansion that stents 104b-104f. As explained in more detail below, with end stents 104a, 104g more resistant to radial expansion than middle stents 104b-104f, a balloon of a balloon catheter will expand the middle stents 104b-104f first, and then a combined radial and axial force will expand end stents 104a, 104g such that foreshortening will be eliminated or minimized. In the embodiment of FIG. 1, ends stents 104a, 104g are more resistant to radial expansion than middle stents 104b-104f by being the same material, but thicker, as can be seen schematically in FIG. 1. In particular, stents 104a, 104g are each at least 2 times more resistant to radial expansion than each of stents 104b-104f. In one particular embodiment, stents 104a, 104g are each approximately 3 times more resistant to radial expansion than each of stents 104b-104f. In an example wherein stainless steel is used for stents 104a-104g, stents 104b-104f each are approximately 0.010 inch in thickness and stents 104a, 104g are each approximately 0.013 inch in thickness. Such an increase in thickness of approximately 0.003 inch in thickness results in approximately a tripling of an increase in resistance to radial expansion. The exemplary thicknesses provided are for stent graft with an expanded diameter of approximately 0.45 inch. Those skilled in the art would recognize that the examples provided above are merely examples for a particular stent graft and do not limit the invention. Accordingly, other materials and thicknesses may be used provided that the end stents have a higher resistance to radial expansion such that foreshortening is reduced or eliminated, as described herein.

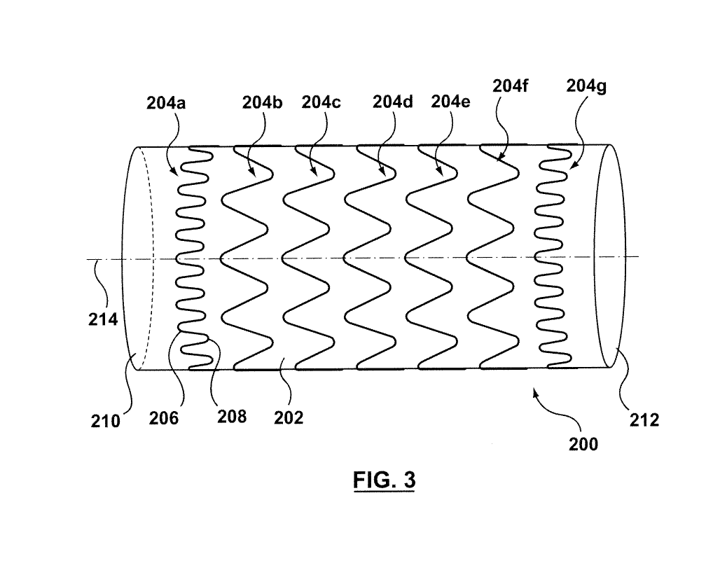

Although FIG. 1 shows that end stents 104a, 104g are made more resistant to radial expansion than middle stents 104b-104f by being thicker, other ways to make the end stents more resistant to radial expansion than the middle stents may also be utilized. For example, and not by way of limitation, FIG. 3 shows schematically a side view of stent graft 200 in accordance with another embodiment hereof. Stent graft 200 includes a graft material 202 and a plurality of stents 204a-204g coupled to graft material 202. Stent graft 200 includes a first end 210 and a second end 212. Stent graft 200 is formed in a tubular shape to form a lumen therethrough and including a longitudinal axis 214, as known in the art.

As discussed above with respect to stent graft 100, stent graft 200 is preferably formed with graft material 202 of expanded polytetrafluoroethylene (hereinafter “ePTFE”) and stents 204a-204g of stainless steel. However, as also explained above, graft material 202 and stents 204a-204g may be formed of other materials. Further, more or less stents 204a-204g may be utilized. As described above, stents 204a-204g are individual rings with a zig-zag or generally sinusoidal shape including a plurality of generally straight segments or struts 206 with adjacent struts connected to each with bends or crowns 208. Further, stents 204a-204g of stent graft 200 are “segmented” in that the stents are not connected to each other except through the graft material 202. In other words, other than the graft material, other structures, such as longitudinal connectors, do not connect the stents 204a-204g to each other.

As described above, stent grafts, and in particular “segmented” stent grafts wherein the stents are not connected to each other, may experience foreshortening when expanded. In the embodiment of FIG. 3, stents 204a and 204g, i.e., the stents adjacent ends 210 and 212 of stent graft 200, are more resistant to radial expansion than stents 204b-204f. As explained in more detail below, with end stents 204a, 204g more resistant to radial expansion than middle stents 204b-204f, a balloon of a balloon catheter will expand the middle stents 204b-204f first, and then a combined radial and axial force will expand end stents 204a, 204g such that foreshortening will be eliminated or minimized. In the embodiment of FIG. 3, rather than making the end stents thicker than the middle stents, end stents 204a, 204g are made more resistant to radial expansion than middle stents 204b-204f by providing more crowns 208 around the circumference of stent graft 200 for stents 204a, 204g than for stents 204b-204f. Increasing the number of crowns 208 generally increases the stents resistance to radial expansion. Further, in the embodiment shown in FIG. 3, struts 206 in end stents 204a, 204g are shorter than struts 206 in middle stents 204b-204f. Although FIG. 3, shows both more crowns 208 and shorter struts in end stents 204a, 204g, those skill in the art would recognize that either or both may be used to make end stents 204a, 204g more resistant to radial expansion than middle stents 204b-204f. It is understood by those skilled in the art that, if the stent is seen as the approximation of a sine curve, any decrease in the amplitude of the strut or increase in the pitch/frequency of the strut pattern will increase the radial force required to expand the stent if the wire diameter remains the same. In this embodiment, if the stent strut of 204a has an amplitude of “A” and the stent strut frequency is “f”, then the corresponding features of 204b would be “A”×(n>1) and/or “f”'(n<1). In particular, as described above, stents 204a, 204g each may be at least 2 times more resistant to radial expansion than each of stents 104b-104f. In one particular embodiment, stents 104a, 104g are each approximately 3 times more resistant to radial expansion than each of stents 104b-104f.

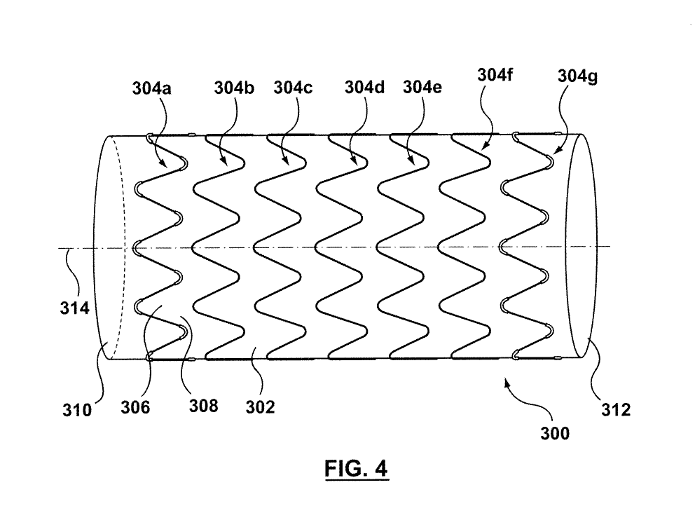

FIG. 4 shows a schematic side view of another embodiment of a stent graft 300 with reduced foreshortening. Stent graft 300 includes a graft material 302 and a plurality of stents 304a-304g coupled to graft material 302. Stent graft 300 includes a first end 310 and a second end 312. Stent graft 300 is formed in a tubular shape to form a lumen therethrough and including a longitudinal axis 314, as known in the art.

As discussed above with respect to stent grafts 100 and 200, stent graft 300 is preferably formed with graft material 302 of expanded polytetrafluoroethylene (hereinafter “ePTFE”) and stents 304a-304g of stainless steel. However, as also explained above, graft material 302 and stents 304a-304g may be formed of other materials. Further, more or less stents 304a-304g may be utilized. As described above, stents 304a-304g are individual rings with a zig-zag or generally sinusoidal shape including a plurality of generally straight segments or struts 306 with adjacent struts connected to each with bends or crowns 308. Further, stents 304a-304g of stent graft 300 are “segmented” in that the stents are not connected to each other except through the graft material 302. In other words, other than the graft material, other structures, such as longitudinal connectors, do not connect the stents 304a-304g to each other.

As described above, stent grafts, and in particular “segmented” stent grafts wherein the stents are not connected to each other, may experience foreshortening when expanded. In the embodiment of FIG. 4, stents 304a and 304g, i.e., the stents adjacent ends 310 and 312 of stent graft 300, are more resistant to radial expansion than stents 304b-304f. As explained in more detail below, with end stents 304a, 304g more resistant to radial expansion than middle stents 304b-304f, a balloon of a balloon catheter will expand the middle stents 304b-304f first, and then a combined radial and axial force will expand end stents 304a, 304g such that foreshortening will be eliminated or minimized. In the embodiment of FIG. 4, rather than making the end stents thicker than the middle stents, end stents 304a, 304g are made more resistant to radial expansion than middle stents 304b-304f by making crowns 308 of end stents 304a, 304g thicker than crowns 308 of middle stents 304b-304f, as shown schematically in FIG. 4. For example, and not by way of limitation, crowns 308 of stents 304a, 304g may be approximately 0.005″ inch thicker than crowns 308 of middle stents 304b-304f. Increasing the thickness of crowns 308 and number of crowns 308 generally increases the stents resistance to radial expansion. In particular, as described above, end stents 304a, 304g are each at least 2 times more resistant to radial expansion than each of stents 304b-304f. In one particular embodiment, end stents 304a, 304g are each approximately 3 times more resistant to radial expansion than each of stents 304b-304f.

Those skilled in the art would recognize that although FIGS. 1, 3, and 4 show different ways to make the end stents more resistant to radial expansion that the middle stents, other ways may be utilized. Further, the embodiments may be combined. For example, and not by way of limitation, the crowns of the end stents may be thicker than the middle stents (as shown in FIG. 4), and the end stents may have a smaller amplitude/greater frequency than the middle stents (as shown in FIG. 3). In such a situation, the combination of features allows each to be not as prominent. For example, the crowns need not be as thick as the embodiment where only the crowns are thicker and the amplitude/frequency remains the same as the middle stents. In a similar fashion, the embodiments of FIGS. 1 and 3 may be combined to make the end stents more resistant to radial expansion than the middle stents.

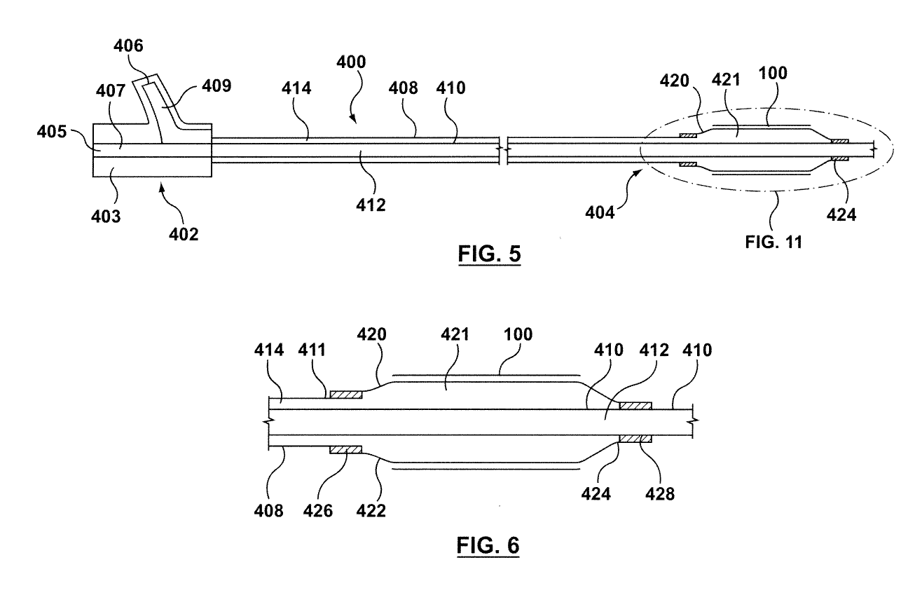

FIGS. 5 and 6 show a balloon catheter 400 with stent graft 100 mounted thereon for delivering stent graft 100 to a desired treatment site and deployed stent graft 100 at the treatment site. Although FIGS. 5 and 6 show stent graft 100 mounted on catheter 400, those skilled in the art would recognize that stent graft 200 or stent graft 300, or variations thereof described above, may be used instead of stent graft 100. As shown generally in FIG. 5, balloon catheter 400 includes a proximal portion 402 and a distal portion 404. Balloon catheter 400 as shown includes an outer shaft 408 and an inner shaft 410 disposed in a lumen of outer shaft 408. A lumen 412 of inner shaft 410 is generally known as a guidewire lumen. An annular or inflation lumen 414 is defined between an outer surface of inner shaft 410 and an inner surface of outer shaft 408. Although a dual shaft or “over-the-wire” balloon catheter is shown, other types of balloon catheters known in the art may be used including, but not limited to, rapid exchange catheters. Proximal portion 402 includes a handle or luer 403, such as a Touhy-Borst adapter. Luer 403 includes an opening 405 for a lumen 407 that is coupled to guidewire lumen 412 of an inner shaft 410. Luer 403 also includes an opening 406 for a lumen 409 that is coupled to inflation lumen 414. Proximal portion 402 may include other devices known to those skilled in the art, such as, but not limited to, strain relief elements, hemodynamic seals, and the like.

Distal portion 404 of balloon catheter 400 is shown in FIG. 5 and in more detail in FIG. 6. A balloon 420 is disposed at distal portion 404 of catheter 400. In the embodiment shown, a proximal portion 422 of balloon is coupled to an outer surface of a distal portion 411 of outer shaft 408 at connection 426 and a distal portion 424 of balloon 420 is coupled to an outer surface of inner shaft 410 at connection 428. Connections 426 and 428 may be an adhesive or other connections know to those skilled in the art. As shown, inner shaft 410 extends distally beyond a distal end of outer shaft 408. Accordingly, inflation lumen 414 extends into an interior 421 of balloon 420, as known in the art. Although a particular embodiment of a balloon catheter is shown, those skilled in the art would recognize that many variations of a balloon catheter may be utilized. Stent graft 100 is mounted around balloon 420, as known in the art. Stent graft 100 is generally radially compressed to a radially compressed configuration for mounting on balloon catheter 400 for delivery to the treatment site.

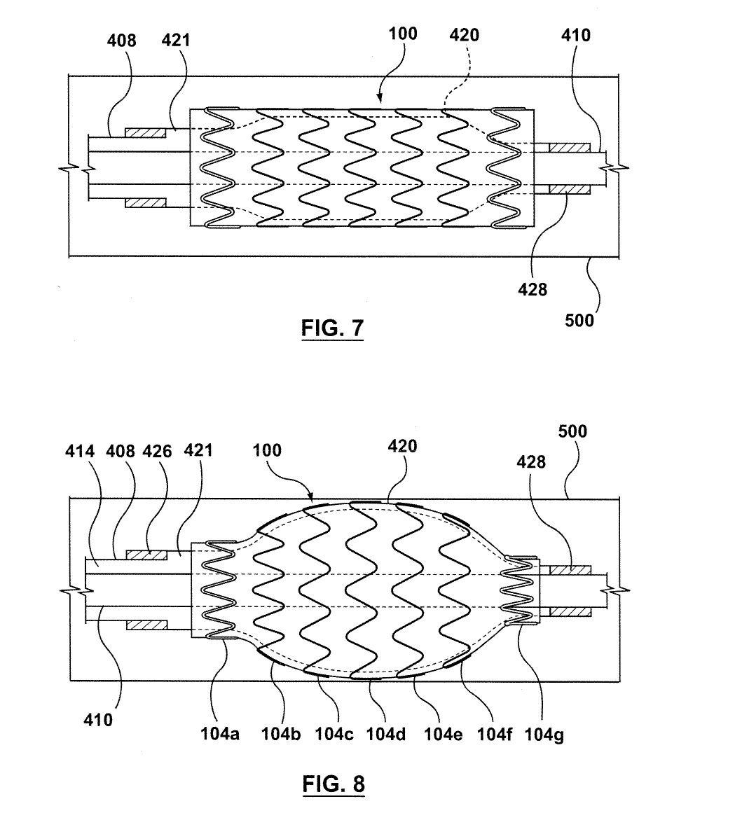

FIGS. 7-11 show a method of deploying stent graft 100 in a vessel 500 with reduced foreshortening. As shown in FIG. 7, catheter 400 is delivered within a vessel 500 to a deployment site. Catheter 400 can be inserted into vessel 500 and delivered through the lumen of vessel 500 by methods known to those skilled in the art. For example, and not by way of limitation, and access opening may be formed into the femoral artery by the Seldinger technique. A guidewire (not shown) may be inserted into vessel 500 and advanced to the deployment site. Catheter 400 may then be advanced along the guidewire to the deployment site, as known in the art. Although shown generally as a vessel 500, segmented stent graft 100 is particularly useful as a branch stent disposed in a branch vessel that is coupled to a main stent graft disposed in a main vessel. For example, and not by way of limitation, end 110 of stent graft 100 may be disposed in a fenestration of a main stent graft deployed in the main vessel, with the remainder of stent graft 100 extending away from the main stent graft and into a branch vessel. For example, and not by way of limitation, the main stent graft may be disposed in the aorta, and stent graft 100 may be disposed in a branch vessel extending from the aorta, such as but not limited to the brachiocephalic artery, the left common carotid artery, the left subclavian artery, and the left and right renal arteries.

Upon reaching the deployment site, a fluid is inserted through opening 406 in luer 403 and into inflation lumen 414. The fluid may be any fluid suitable for use in inflatable a balloon, such as, but not limited to, a saline solution. The inflation fluid travels from inflation lumen 414 into interior 421 of balloon 420. As interior 421 fills, balloon 420 expands, as shown in FIG. 8. Because end stents 104a, 104g have a higher resistance to radial expansion, middle stents 104b-104f begin to expand prior to end stents 104a, 104g, as shown schematically in FIG. 8.

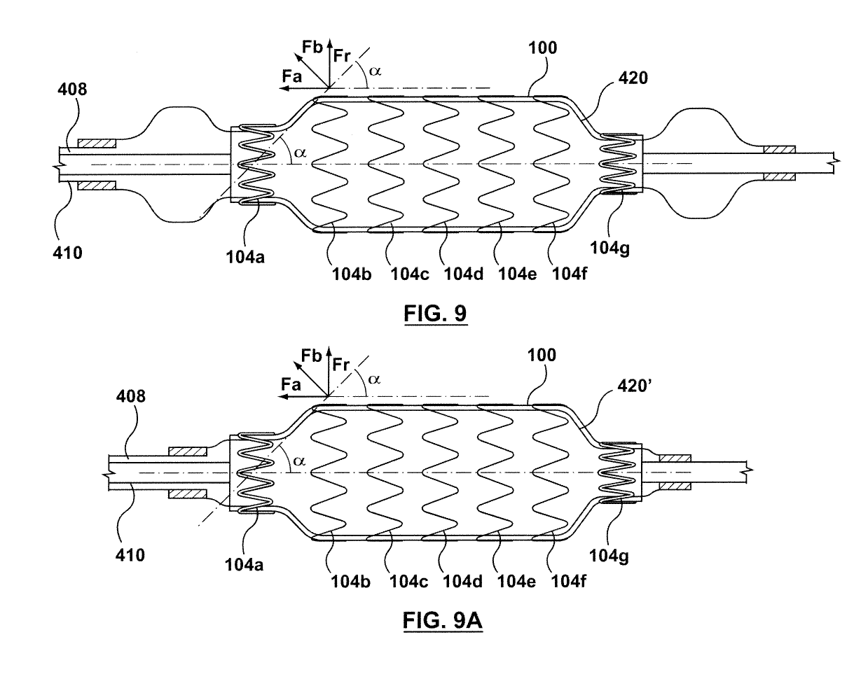

As the expansion fluid continues to fill interior 421 of balloon 420, the outward radial pressure of balloon 420 increases. FIG. 9 shows a schematic view of balloon 420 expanding with stent graft 100 disposed thereon with vessel 500 other details of catheter 400 not included for clarity. As can be seen schematically in FIG. 9, middle stents 104b-104f have been expanded by balloon 420. However, due to their increased resistance to radial force, end stents 104a, 104g have not been expanded. As middle stents 104b-104f are expanding, but end stents 104a, 104g are not, a balloon force F_{b} is exerted by balloon 420 on end stents 104a, 104g. Balloon force F_{b} includes a radial component Fᵣ which tends to expand end stents 104a, 104g radially outwardly, and an axial component Fₐ which tends to push ends stents 104a, 104g axially away from each other. This axial component force Fₐ prevents end stents 104a, 104g from collapsing, i.e. “train wrecking” towards middle stents 104b-104f, thereby maintaining the desired length of stent graft 100. In the embodiment of FIG. 9, balloon 420 is longer than stent 100.

In another, embodiment, shown in FIG. 9A, a balloon 420′ is approximately the same length as the desired length for stent 100 when deployed. As can be seen in FIG. 9A, as expansion fluid fills interior 421′ of balloon 420′, the outward radial pressure of balloon 420′ increases. Similar to FIG. 9, middle stents 104b-104f have been expanded by balloon 420′. However, due to their increased resistance to radial force, end stents 104a, 104g have not been expanded. As middle stents 104b-104f are expanding, but end stents 104a, 104g are not, a balloon force F_{b} is exerted by balloon 420′ on end stents 104a, 104g. Balloon force F_{b} includes a radial component Fᵣ which tends to expand end stents 104a, 104g radially outwardly, and an axial component Fₐ which tends to push ends stents 104a, 104g axially away from each other. This axial component force Fₐ prevents end stents 104a, 104g from collapsing, i.e. “train wrecking” towards middle stents 104b-104f, thereby maintaining the desired length of stent graft 100. In the embodiment of FIG. 9A, because balloon 420′ is approximately the same length of stent 100, no inward axial forces from the portions of balloon 420 outside stent graft 100 (as shown in FIG. 9) act on stent graft 100.

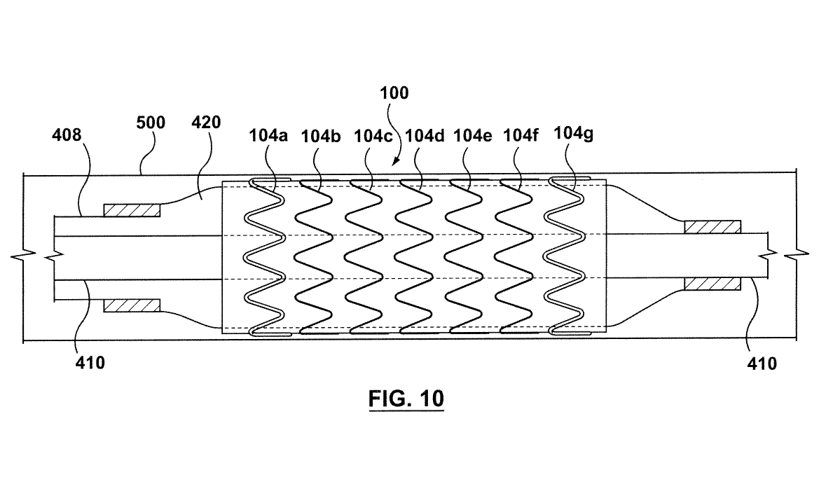

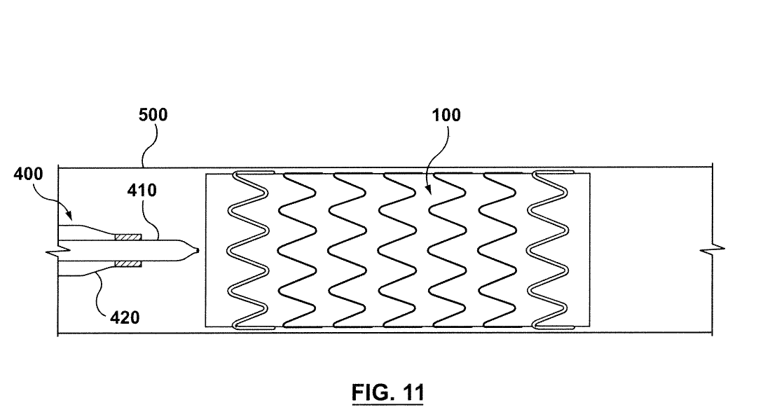

As the fluid pressure within balloon 420, 420′ increases, thereby increasing the pressure exerted by balloon 420, 420′ on stent graft 100, the increased resistance to radial force of end stents 102a, 102g is overcome, thereby expanding end stents 102a, 102g, as shown in FIG. 10. With stent graft 100 deployed, the inflation fluid may be withdrawn from interior 421, 421′ of balloon 420, 420′ and catheter 400 may be removed, leaving stent graft 100 deployed in vessel 500, as shown in FIG. 11.

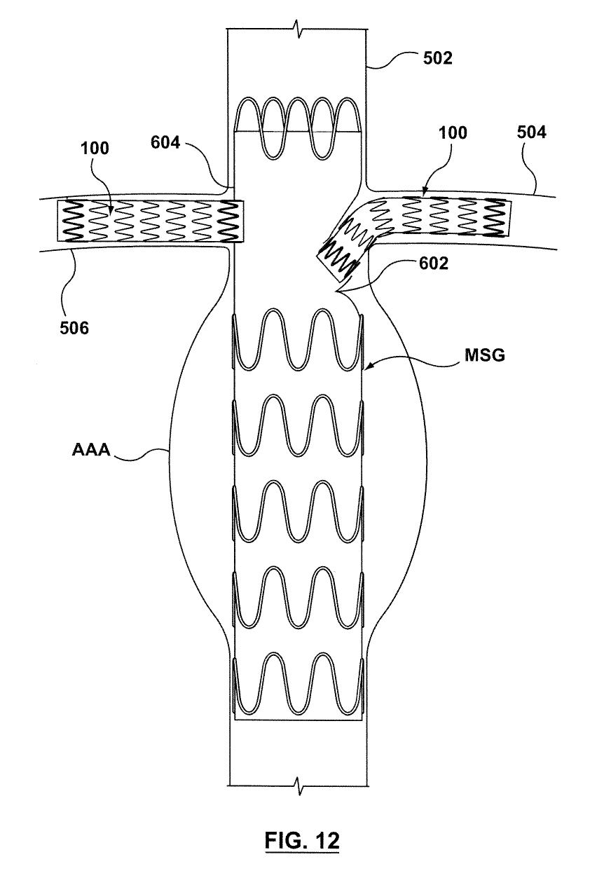

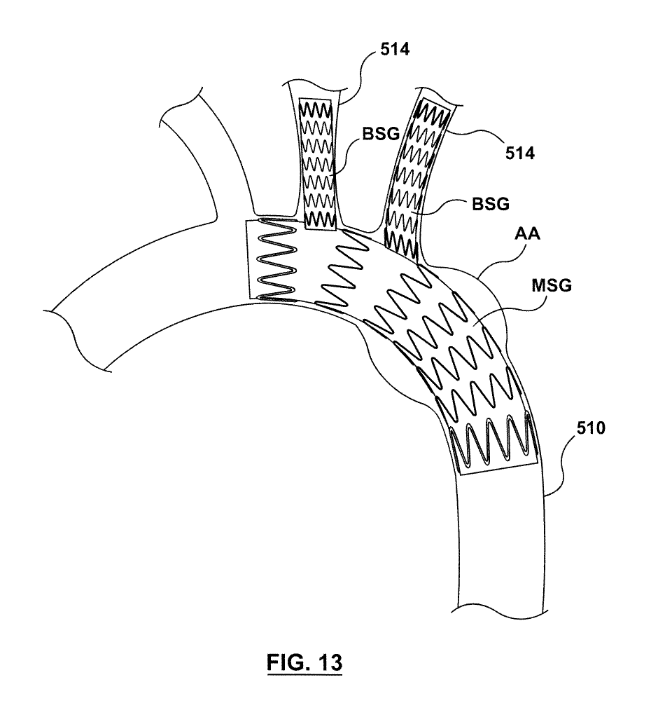

FIGS. 12 and 13 show potential uses of the stent grafts described herein in different locations. These locations are only examples and do not limit the potential uses and locations for the stent grafts described herein. FIG. 12 shows a main stent graft MSG disposed in the descending aorta 502 at the site of an abdominal aortic aneurysm AAA. In the embodiment shown, main stent graft MSG is a conventional stent graft including fenestrations or apertures 602, 604 for access to the renal arteries 504, 506. In the embodiment shown, branch stent grafts 100 are disposed in fenestrations 602, 604 and into renal arteries 504, 506. The branch stent grafts 100 are as described herein. Although reference numeral 100 is used, those skilled in the art would recognize that any of the stent graft described herein can be used, including variations described herein. In the embodiment of FIG. 12, one of the fenestrations 602 is not aligned with renal artery 504. Accordingly, branch stent graft 100 extends from fenestration 602 is the main vessel 502 and has to bend/curve to gain access to renal artery 504. Further, due to the large volume of blood pumped through the aorta, the aorta moves significantly. Thus, a highly flexibly stent graft, without foreshortening, as described herein, is desirable.

FIG. 13 shows another exemplary use of the stent graft described herein. In the embodiment of FIG. 13, a main stent graft MSG is disposed in the aortic arch 510 having an aortic aneurysm AA. Further, branch stent grafts BSG extend from fenestrations in the main stent graft MSG and into branch arteries 512, 514. In the embodiment shown, the branch arteries 512, 514 are the left common carotid artery and the left subclavian artery, respectively. However, those skilled in the art would recognize that the main stent graft MS may be located in other areas and more or less branch arteries may be involved. In the embodiment shown in FIG. 13, the main stent graft MSG and branch stent grafts BSG are each stent graft as described herein. In other words, main stent graft MSG and branch stent grafts BSG are balloon expandable segmented stent grafts with end stents more resistant to radial expansion than middle stents, as described above. Although the stent grafts of FIG. 13 are shown with the embodiment of FIG. 1, any of the embodiments described herein, and variations thereof, may be utilized. Further, although all of the stent grafts of FIG. 13 are shown to be stent graft according to the descriptions herein, less than all of the stent grafts can be stent grafts of the present application. For example, the main stent graft MSG may be a conventional stent graft and one or both of the branch stent grafts BSG may be stent grafts of the present application. Similarly, the main stent graft MSG may be a stent graft of the present application and one or both of the branch stent grafts BSG may be a conventional stent graft. With the curvature of the aortic arch and the motion thereof, it is desirable to have highly flexible stent grafts disposed in the area, while simultaneously limiting foreshortening, as disclosed in the present application.

While various embodiments have been described above, it should be understood that they have been presented only as illustrations and examples of the present invention, and not by way of limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A balloon expandable stent graft comprising:
a graft material of generally tubular configuration; and
a plurality of cylindrical stent elements coupled to the graft material, wherein the plurality of cylindrical stent elements are plastically deformable when expanded from a radially compressed configuration to a radially expanded configuration, wherein the plurality of cylindrical stent elements include,
a first end stent element disposed adjacent a first end of the graft material;
a second end stent element disposed adjacent a second end of the graft material; and
a plurality of middle stent elements disposed between the first end stent element and the second end stent element,
wherein the first end stent element is independent of the plurality of middle stent elements and the second end stent element is independent of the plurality of middle stent elements, and
wherein the first end stent element and the second end stent element are more resistant to radial expansion than the plurality of middle stent elements such that the plurality of middle stent elements plastically deform from the radially compressed configuration to the radially expanded configuration when inflated by a balloon prior the first end stent element and the second end stent element.

2. The balloon expandable stent graft of claim 1, wherein the first end stent element and the second end stent element are at least twice as resistant to radial expansion as the middle stent elements.

3. The balloon expandable stent graft of claim 1, wherein the first end stent element and the second end stent element are at least three times as resistant to radial expansion as the middle stent elements.

4. The balloon expandable stent graft of claim 1, wherein the plurality of middle stent elements are independent of each other.

5. The balloon expandable stent graft of claim 1, wherein each of the plurality of cylindrical stent elements comprises a ring having a plurality of struts and a plurality of crowns connecting the struts to each other.

6. The balloon expandable stent graft of claim 5, wherein the struts of the first end stent element and the struts of the second end stent element are thicker than the struts of the plurality of middle stent elements.

7. The balloon expandable stent graft of claim 6, wherein the crowns of the first end stent element and the crowns of the second end stent element are thicker than the crowns of the plurality of middle stent elements.

8. The balloon expandable stent graft of claim 1, wherein the crowns of the first end stent element and the crowns of the second end stent element are thicker than the crowns of the plurality of middle stent elements.

9. A method of deploying a stent graft in a vessel comprising the steps of:
delivering the stent graft to a site within the vessel with the stent graft in a radially compressed configuration, wherein the stent graft includes a first end portion, a second end portion, and a middle portion disposed between the first end portion and the second end portion, the stent graft comprising a graft material and a plurality of independent stent elements coupled to the graft material; and
radially expanding the stent graft by applying a substantially uniform radial pressure to the stent graft, wherein the independent stent elements disposed at the first end portion of the stent graft and the independent stent elements disposed at the second end portion of the stent graft expand at a higher radial pressure than the independent stent elements disposed at the middle portion of the stent graft such that the middle portion of the stent graft expands before first end portion and the second end portion.

10. The method of claim 9, wherein the step of radially expanding the stent graft comprises inflation a balloon disposed within a lumen of the stent graft.

11. The method of claim 10, wherein the balloon is inflated to a first pressure such that the middle portion of the stent graft expands while the first end portion and the second end portion do not expand, wherein the balloon continues to be inflated to a second pressure such that the first end portion and the second end portion expand.

12. The method of claim 11, wherein the second pressure is at least twice the first pressure.

13. The method of claim 11, wherein the second pressure is at least three times the first pressure.

14. The method of claim 9, wherein each of the plurality of independent stent elements comprises a ring having a plurality of struts and a plurality of crowns connecting the struts to each other.

15. The method of claim 14, wherein the struts of the independent stent elements at the first end portion and the struts of the independent stent elements at the second end portion are thicker than the struts of the independent stent elements at the middle portion.

16. The method of claim 15, wherein the crowns of the independent stent elements at the first end portion and the crowns of the independent stent elements at the second end portion are thicker than the crowns of the independent stent elements at the middle portion.

17. The method of claim 9, wherein the crowns of the independent stent elements at the first end portion and the crowns of the independent stent elements at the second end portion are thicker than the crowns of the independent stent elements at the middle portion.
